(19) United States
(12) Patent Application Publication
(10) Pub. No.: US 2003/0059797 A1
(43) Pub. Date: Mar. 27, 2003

(54) Animal model of and test for Alzheimer's disease

(76) Inventor: Elena Paley, Surfside, FL (US)

Correspondence Address: Mark G. Lappin, Esq., P.C., McDERMOTT, WILL & EMERY, 28 State Street, Boston, MA, 02109, US

(21) Appl. No.: 10/126,467

(22) Filed: Apr. 19, 2002

Related U.S. Application Data
(63) Continuation-in-part of application No. 09/513,895, filed on Feb. 28, 2000, now abandoned.
Application No. 09/513,895 is a continuation-in-part of application No. 09/384,869, filed on Aug. 27, 1999, now Pat. No. 6,221,662.
(60) Provisional application No. 60/284,980, filed on Apr. 19, 2001.

(51) Int. Cl. C12N 5/00; C12N 5/02; C12Q 1/68; G01N 33/53

(52) U.S. Cl. 435/6; 435/325; 435/7.1

(57) ABSTRACT

Tryptamine, a normal constituent of the diet and an inhibitor of tryptophanyl-tRNA synthetase (TrpRS), induces formation of Alzheimer's disease characteristics as extracellular senile plaques and neurofibrillary tangles containing paired helical neurofilaments and/or straight neurofilaments in human differentiated neuronal cells. The effect of tryptamine on neuronal cells is dose-dependent and multi-staged resulting in differentiation and subsequently axon growth, followed by neurodegeneration. TrpRS was found normally in the neuronal body, axons and dendrites, whereas tryptamine induces it in the cytoskeleton. In tryptamine-treated cells, TrpRS co-localized with abnormally phosphorylated tau in neurofibrillary tangles and with β-amyloid in senile plaque-like formations. Moreover, TrpRS was detected in neurofibrillary tangles and congophilic senile plaques in Alzheimer's disease brain sections and brain sections of model mice following tryptamine treatment. The secreted TrpRS was demonstrated in human sera. These findings provide a new perspective on the versatility of TrpRS functions and indicate that tryptamine may be involved in the induction of Alzheimer's disease.

### FIELD OF THE INVENTION

This invention relates to development of a test for Alzheimer's disease based on a biological marker, which test detects and identifies morphological indicators for Alzheimer's disease.

### BACKGROUND OF THE INVENTION

Tryptophanyl-tRNA synthetase (TrpRS) is an enzyme that catalyzes the attachment of tryptophan to tRNA^{trp} in the protein translation. The mammalian enzyme is a phosphoprotein present in the cytoplasm, mitochondrion, nucleus and cytoskeleton. Paley et al., Exp. Cell Res. 195: 66-78 (1991); Paley, Eur. J. Biochem. 244: 780-788 (1997); Paley, Cancer Lett. 137: 1-7 (1999). In contrast to other members of the aminoacyl-tRNA synthetases (ARS) family, TrpRS expression is induced by interferons.

Continuous treatment of bovine kidney cell line with inhibitors of TrpRS (such as tryptamine and tryptophanol) resulted in plural alterations, including formation of tangles of filaments with a diameter of 15-20 nm possessing TrpRS. Paley et al., Exp. Cell Res. 195: 66-78 (1991). These tangles are similar to the neurofibrillary tangles (NFT) that characterize Alzheimer's disease (AD) brain. The Alzheimer's disease intracellular neurofibrillary tangles consist of paired helical filaments or straight filaments of 8-20 nm in diameter containing hyperphosphorylated tau.

Alzheimer's disease is a progressive neurodegenerative disease that affects an estimated four million people throughout the United States alone and is characterized by progressively worsening memory loss and eventual dementia. Currently, one in ten people over the age of 65 suffer from some stage of the disease. In addition, half of all people over the age of 85 demonstrate some symptomatic signs of Alzheimer's disease.

Currently, the definitive diagnosis for Alzheimer's disease can only be made after a patient's death, when the autopsied brain tissues can be studied. Doctors and medical researchers examine patients for evidence of memory and thinking difficulties, symptoms that present only once the disease has advanced. Moreover, other recognized tools for the determination of the existence of Alzheimer's disease include CAT scans and magnetic resonance imaging (MRI). Both of these techniques, however, only show damage to the brain cells once the Alzheimer's disease has taken a firm hold.

Accordingly, there is a need in the medical field for new advancements in research techniques and procedures, for a simple test to identify biological markers for Alzheimer's disease.

### SUMMARY OF THE INVENTION

This invention provides a test for Alzheimer's disease, based on detecting morphological indicators of Alzheimer's disease, including senile plaques and neurofibrillary tangles. Anti-TrpRS monoclonal and polyclonal antibodies recognize neurofibrillary tangles and senile plaques in human brain sections, based upon recognition of the presence of TrpRS in these Alzheimer's disease morphological indicators. Secreted TrpRS is also demonstrated in human sera and other fluids.

The invention also provides an animal model of Alzheimer's disease, produced by administering tryptamine to the animals. Tryptamine is a natural metabolite and an inhibitor of tryptophanyl-tRNA synthetase (TrpRS). Administration of tryptamine to the animals induces formation of Alzheimer's disease characteristics, including the morphological and behavioral characteristics similar to those found in Alzheimer's disease patients.

The invention further provides a method for screening drugs that interact with neurons containing the characteristics of Alzheimer's disease.

### BRIEF DESCRIPTION OF THE DRAWINGS

FIG. 1 shows tryptamine-induced neurodifferentiation. FIG. 1a is a confocal micrograph of tryptamine-treated cells labeled with antibodies to synaptophysin. FIG. 1b shows peroxidase labeling with antibodies to nse (neuron-specific enolase). FIG. 1c shows peroxidase labeling with antibodies to nf (neurofilament). FIG. 1d shows peroxidase labeling with antibodies to map (microtubule-associated protein 2). FIG. 1e shows peroxidase labeling with antibodies to wrs (anti-TrpRS). FIG. 1f is a phase contrast micrograph of terminally differentiated neurons (upper right), and long-term cultivated neuron-like clone (low). Arrow shows neuroblast near to neuron (upper left). Bar=7 m.

FIG. 2 shows tryptamine-induced neurodegeneration. FIG. 2a shows peroxidase staining of the treated cells with polyclonal antibody to TrpRS. FIG. 2b shows peroxidase staining of the treated cells with antibody to AT8. FIG. 2c shows staining of cells with Congo Red. FIG. 2d shows monoclonal antibody (mAb) to -amyloid 508 and 10D5. FIG. 2e-FIG. 21 show fluorescent labeling of treated (FIG. 2e, f, i, and j) cells and control (FIG. 2h and FIG. 2k) cells with polyclonal antibody TrpRS/AT8 (FIG. 2e, g, and h), TrpRS/tau (FIG. 2f), anti-TrpRS 6C10/C9 (FIG. 2i, j, and k). FIG. 21 is a control developed with secondary antibody. Red is tau, green, TrpRS. FIG. 2m is an electron micrograph of tryptamine-treated cells labeled with anti-TrpRS (WRS) and AT8 following by development with immunogold of 10 nm or processed without immunodetection. FIG. 2a-d, Bar=26 m; FIG. 2m, Bar=170 nm.

FIG. 3 is a set of Western blots of tau protein and TrpRS. In FIG. 3a, tau protein detected with AT8 in the total extracts (right panels) and detergent-insoluble fractions (left lanes) of the control (C) and treated (T) cells. Two right lanes are overexposed middle lanes (total). In FIG. 3b, bovine (B) and human recombinant (H) TrpRS detected with polyclonal (p) and 6C10/C9 (m) antibodies. In FIG. 3c, TrpRS in total and detergent-insoluble fractions of control and treated cells detected with 6C10/C9. Western blots of the middle and right panels were conducted after stripping the blots probed with AT8. Left panel shows non-re-probed blot. Molecular weight markers in kilodaltons (kDa) are on the side.

FIG. 4 shows TrpRS in the brain and sera. FIG. 4a is a micrograph showing I-NFT stained with 9D7. FIG. 4b is a micrograph showing double staining with anti-tau/TrpRS polyclonal antibody. FIG. 4c is a micrograph showing I-NFT stained with anti-TrpRS polyclonal antibody. FIG. 4d shows neurons stained with anti-TrpRS polyclonal antibody (from a non-demented control individual). FIG. 4e-FIG. 4h show E-NFT stained with anti-TrpRS polyclonal antibody (upper panel of FIG. 4e and left panel of FIG. 4g) and double labeled with anti-TrpRS/ -amyloid 508 (low panel of FIG. 4e; right panel of FIG. 4g; and panels FIG. 4f and FIG. 4h). FIG. 4h and FIG. 4I are confocal microscopy micrographs of E-NFT (FIG. 4h) and double-labeled neurons (FIG. 4i). FIG. 4j-FIG. 4l are micrographs showing senile plaque stained with anti-TrpRS polyclonal antibody and monoclonal antibody (mAb) 6C10/C9 (FIG. 4j, upper panels) and then counterstained with Congo Red/hematoxylin (FIG. 4k, FIG. 4l). FIG. 4k (bottom panel) shows the birefringence of senile plaques shown in FIG. 4k (upper panel). FIG. 4l is a confocal microscopy micrograph of the senile plaques in FIG. 4k (Upper panels show fluorescence; lower panels show phase contrast; right panels are enlarged optical sections of the left). FIG. 4m shows senile plaques labeled with anti-TrpRS polyclonal antibody (upper) and double-labeled with anti-TrpRS/ -amyloid 508 (lower). FIG. 4n shows vessel staining with anti-TrpRS polyclonal antibody (upper left), double labeling with anti-TrpRS/ -amyloid 508 (upper right), counterstaining with Congo Red/hematoxylin (lower left) and birefringence (right low). FIG. 4o is an immunoblot of human sera with 6C10/C9. Serum albumin and TrpRS (wrs) are shown on the right. Except for FIG. 4d, the neurons are from subjects with Alzheimer's disease. Arrows show I-NFT (black) in FIG. 4a-FIG. 4c; undamaged neurons in FIG. 4a, FIG. 4b, and FIG. 4d; and neuropil threads (curly fibers) in FIG. 4j (white). Bar=35 m.

FIG. 5 is a set of micrographs showing Congo Red staining and birefringence of sagittal sections from brains of tryptamine-treated mice. The arrow shows Congo Red-stained plaque in hippocampus, with the arrowhead pointing to identical deposits in Congo Red staining and birefringence.

FIG. 6 is a set of micrographs showing silver staining of Alzheimer's neurofibrillary changes in the brain sections of tryptamine-treated mice.

FIG. 7 is a set of tables and graphs showing measurements of the treated mice's behavior. The control and tryptamine-treated model mice were put on a narrow 1-cm diameter metallic stick. The time that mice balanced on the stick was measured in seconds. 66% of control versus 10% of treated mice kept on the stick for more than 6 seconds in the first experiment and 66% of control versus 20% of treated were for more than 13 seconds in the second experiment. Thus, the model mice were 6.6 times less balanced or coordinated than control.

### DETAILED DESCRIPTION

To date, there has been no evidence that neurofibrillary tangles are present in either neuronal cell lines or animals. Cell treatment with the phosphatase inhibitor, okadaic acid, results only in tau hyperphosphorylation, but not neurofibrillary tangles. Tanaka et al., FEBS Lett. 426: 248-254 (1998). Neither cellular nor animal models have been reported to exhibit both main Alzheimer's disease hallmarks: neurofibrillary tangles (NFT) and senile plaques (SP).

By this invention, however tryptamine induces formation of Alzheimer's disease characteristics as extracellular senile plaques and neurofibrillary tangles containing paired helical (PHF) and/or straight (SF) neurofilaments in human differentiated neuronal cells. Tryptamine also induces formation of Alzheimer's disease characteristics in animals, to create an animal model of Alzheimer's disease. Tryptamine is a biogenic amine, decarboxylated analog of tryptophan. Tryptamine is a normal constituent of the diet and an inhibitor of tryptophanyl-tRNA synthetase (TrpRS). Once tryptamine is consumed, it easily crosses the blood-brain barrier. Providing a tryptamine-induced cellular and animal model expressing Alzheimer's infected characteristics is a physiologically useful tool to the investigation of the cause and treatment of Alzheimer's disease.

The effect of tryptamine is dose-dependent and multi-staged, resulting in differentiation and subsequently axon growth, followed by neurodegeneration. TrpRS was found normally in the neuronal body, axons and dendrites, whereas tryptamine induces it in the cytoskeleton. In tryptamine-treated cells, TrpRS co-localized with abnormally phosphorylated tau in neurofibrillary tangles and with p-amyloid in senile plaque-like formations.

To test for the presence of TrpRS, anti-TrpRS antibodies are used. Mammalian TrpRS is preferably used to raise anti-TrpRS antibodies in mice and rabbits. In particular, rabbit polyclonal antibodies were raised against purified bovine TrpRS (fraction G-100) (Paley, Eur. J. Biochem. 244: 780-788 (1997); see EXAMPLE I). These monospecific polyclonal antibodies react with both bovine and human TrpRS under non-denaturing conditions of ELISA, dot-blot and immunohistochemistry. Thus, the antibody specificity can be verified by ELISA, immnunoblotting and immunocytochemistry, using TrpRS that is biochemically isolated from mammalian tissue and recombinant human TrpRS expressing in E coli (see EXAMPLE I). In addition, two distinct mouse IgG₁ monoclonal antibodies (mAb; clones 6C10/C9 and 9D7) were raised to purified bovine TrpRS (Fractogel fraction; Paley, Eur. J. Biochem. 244: 780-788 (1997)) and generated as hybridoma medium and ascites fluids (see EXAMPLE I). The two distinct monoclonal mouse antibodies can be used for immunohistochemical analysis.

Various samples can be analyzed with the antibodies, including brain tissue sections of Alzheimer's disease patients, non-demented patients, mice, tryptamine-treated human differentiated neuronal Alzheimer-like cells, and untreated control cells. The antibodies can be used for peroxidase or peroxidase-anti peroxidase immunolabeling assays, followed by comparisons using Congo Red staining or using double labeling assays with antibodies to beta amyloid protein or tau proteins. Additionally, hematoxylin can be used for counter-staining and to serve as a baseline or comparison model.

The anti-TrpRS antibodies immunostain different morphologically types of plaques, intracellular and extracellular neurofibrillary tangles, congophilic blood vessels and neuropil threads in brain tissue sections of Alzheimer's disease patients, due to the anti-TrpRS antibodies reacting with or attaching to the TrpRS present therein. The detected presence of TrpRS in other areas of the tissue samples (where TrpRS is normally found) provides an internal control for the elevated or abnormal of TrpRS in the Alzheimer's patient. Also, anti-TrpRS antibodies can also be used in staining tryptamine treated differentiated neuronal human Alzheimer-like cells, as an external control to the Alzheimer's patient specimen.

Once the anti-TrpRS antibodies have been administered, these primary antibodies can either be labeled by themselves or detected by labeled secondary antibody. A secondary antibody to the selected monoclonal or polyclonal anti-TrpRS antibody can typically be generated or identified utilizing conventional procedures. For example, if a mouse TrpRS antibody is used, a corresponding secondary antibody that is particularly configured to attach with and stain the TrpRS antibody is used. Accordingly, an indirect staining is effectively achieved revealing the presence of the biological marker, which in the preferred embodiment is TrpRS, and identifies the tangles and plaques. The secondary antibodies used in EXAMPLE I were peroxidase-conjugated goat anti-mouse IgM, peroxidase-conjugated F(ab′)₂ fragment goat anti-mouse or anti-rabbit IgG F(ab′)₂ fragment specific, F(ab′)₂ peroxidase-anti-peroxidase (PAP) produced in mice, peroxidase-conjugated goat anti-mouse or anti-rabbit IgG (for blots), Cy 3-conjugated F(ab′)₂ fragment goat anti-mouse IgG F(ab′)₂ fragment specific, BSA (IgG-free, protease-free) (Jackson IRL, PA, USA), FITC-conjugated goat anti-rabbit IgG (Sigma, USA), 10 nm colloidal gold goat anti-mouse or anti-rabbit IgG (Zymed, CA, USA).

In addition, Congo Red staining can be used to show staining of TrpRS deposits in both senile plaque and cerebral vessels. The structures exhibit an apple-green birefringence, the coloration being indicative of the presence of TrpRS in congophillic deposits.

Accordingly, the invention provides a test kit for the detection of Alzheimer's disease based on the morphological indicators of intracellular and extracellular neurofibrillary tangles and senile plaque in human brain tissue, which are detectable by targeting ARS, as well as monospecific antibodies against ARS. Because of the presence of TrpRS as a constituent of ARS, providing TrpRS monoclonal and polyclonal antibodies can be used for the detection of Alzheimer's characteristics in human brain tissue and would serve to identify the ARS. The kit is useful for the detection of characteristic tangles or plaques by targeting aminoacyl-tRNA synthetase (ARS), through the use of monospecific antibodies against ARS, specifically, as antibodies to tryptophanyl-tRNA synthetase (TrpRS). Recombinant human TrpRS is added as a positive control. Tryptamine-treated human neuronal differentiated Alzheimer-like cells provide a cell culture spent medium that can also be added as a positive control to test for secretion of TrpRS into a medium.

Based on the above, various patient body fluids (sera, plasma, cerebrospinal fluid and other patient fluid) can be tested for the presence of TrpRS and its fragments with anti-TrpRS antibodies using different immuno-techniques including ELISA, immunoblotting, etc. to accomplish effective identification of Alzheimer's disease.

Moreover, the cellular model of the invention can be used to screen for drugs that can effectively alleviate the characteristics of Alzheimer's disease.

The following EXAMPLES are presented in order to more fully illustrate the preferred embodiments of the invention. These examples should in no way be construed as limiting the scope of the invention, as defined by the appended claims.

### EXAMPLE I

TrpRS-Mediated Alzheimer's Disease Hallmarks Induced in Human Neurons by Tryptamine Treatment

The involvement of TrpRS inhibitors in neurofibrillary tangles formation was tested in the human neuroblastoma SH SY5Y cell line expressing tau. Tanaka et al., FEBS Lett. 426: 248-254 (1998). This cloned line comprises two morphologically distinct cell types: predominantly neuroblastic (bearing short neurites) and epithelial-like (lacking neurites). Biedler et al., Cancer Res. 33: 2643-2652 (1973). SH SY5Y cells were grown in 25 cm² flasks (TPP, Switzerland) in 5% CO₂ at 37° C. in RPMI 1640 medium supplemented with 10% fetal calf serum, 100 u/ml penicillin, 100 μg/ml streptomycin, 0.25 μg/ml amphoterycin and 2 mM glutamine. The neuron/axon lines were isolated after seeding microscopically verified single epithelial cells.

Following cultivation with 50-100 μg/ml tryptamine or tryptophanol for 5 days at initial density of 1×10³ on 10-cm dishes all cells have died. Continuous treatment with lower concentrations of the inhibitors led to accumulation of large flattened, tightly substrate adherent epithelial-like cells lacking neurites, while the majority of neuroblasts died. The epithelial cells being cultivated further without inhibitors gave rise to neurites outgrowth showing morphology of unipolar, bipolar or multipolar neurons possessing the axon-like processes (more than twenty-fold longer than neurites of neuroblasts) often terminated in elaborate growth cones.

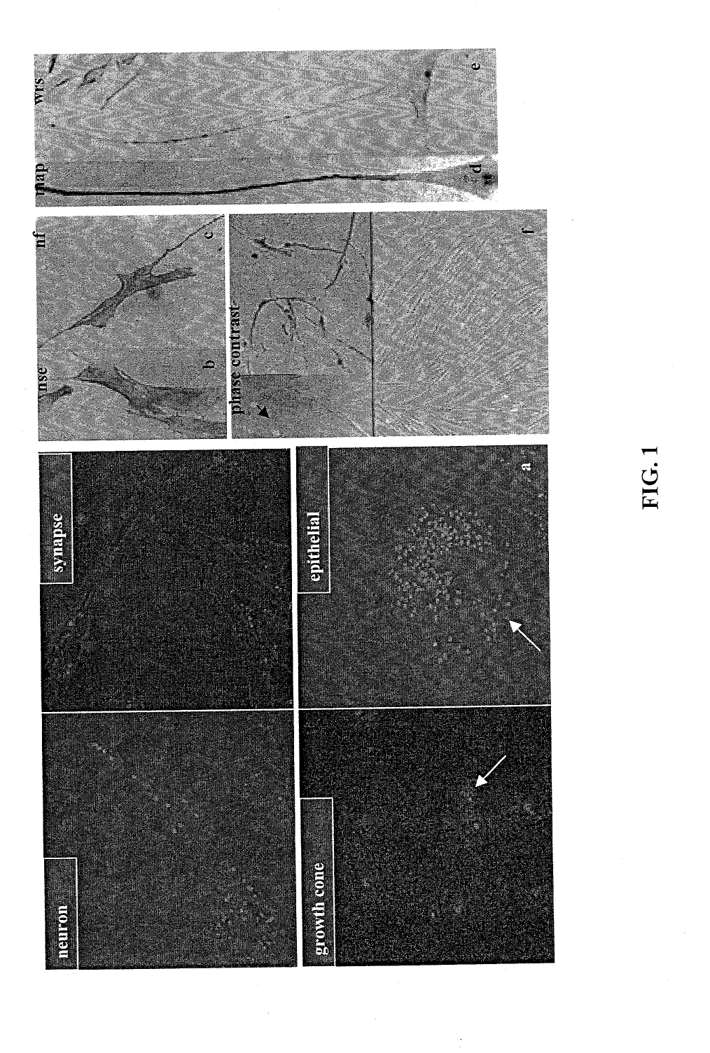

FIG. 1a shows that the phenotypically neurons express synaptophysin, a synaptic vesicle marker. Anti-synaptophysin mAb, clone SY38 was obtained from ICN Biomedicals Inc. (Ohio, USA). Anti-GFAP mAb cocktail was obtained from PharMingen (CA, USA). The neuronal origin of the tryptamine-generated cells was confirmed by detecting neuron-specific enolase (nse, FIG. 1b); neurofilaments (nf, FIG. 1c) and microtubule-associated protein 2 (map, FIG. 1d). Neural cell type identification antibody kit, anti-MAP2 mouse mAb and rabbit polyclonal antibody to human brain neuron-specific enolase were obtained from Calbiochem-Novabiochem (US).

TrpRS was mainly localized in axons and to a less extent in the neuronal body and dendrites (wrs, FIG. 1e). Following cell growth for 4-6 weeks without inhibitors, neuroblasts, being survived or “interconverted” from epithelial cells as earlier suggested by Biedler et al., Cancer Res. 33: 2643-2652 (1973), have appeared in the majority of epithelial/neuron lines and eventually overgrew them. In some sublines, however no neuroblasts were observed during 12 months cultivation without inhibitors (low panel, FIG. 1f). These results indicate that (i) tryptamine is more toxic for neuroblasts than for epithelial cells, (ii) tryptamine induces neurodifferentiation of the epithelial cells and (iii) TrpRS presents preferably in axons of neurons.

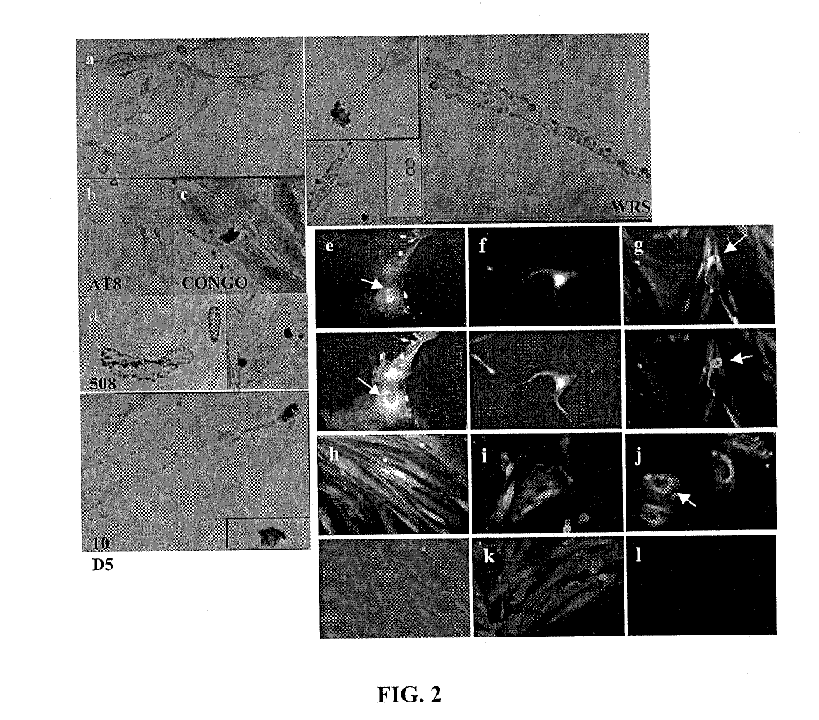

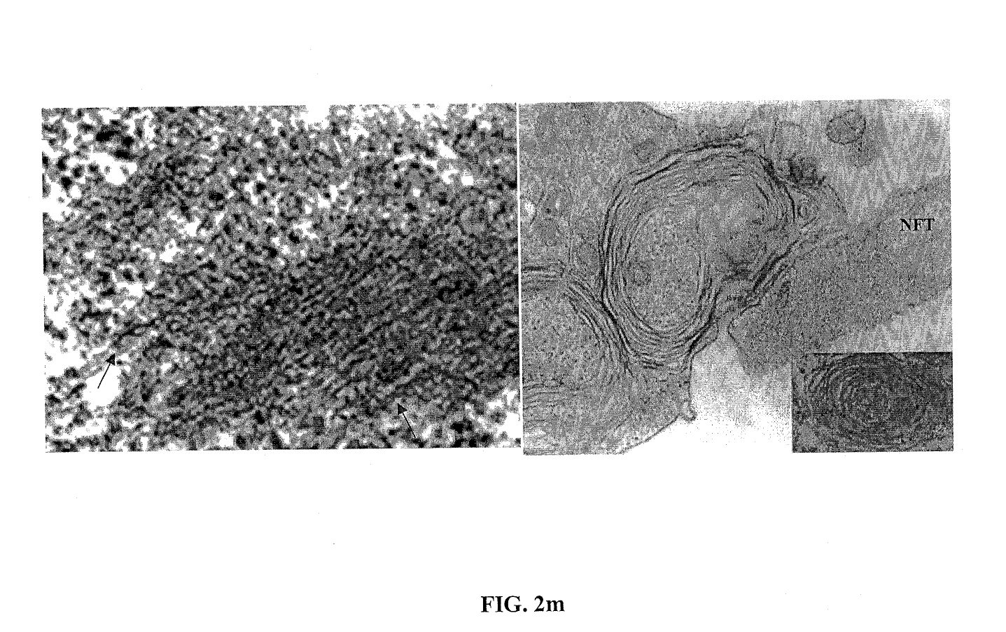

To examine control epithelial cells, which were not pretreated with inhibitors, neuroblastic and epithelial-enriched cells were separated from one another by exploiting a marked difference in adhesive properties. Biedler et al., Cancer Res. 33: 2643-2652 (1973). The weakly adherent neuroblasts were washed out with trypsin. The selected epithelial-enriched and tryptamine-differentiated neuron-axon cells were treated with 20-100 μg/ml tryptamine for 6-60 days and then stained. Peroxidase staining of manifestations resembling intracellular (I), extracellular (E) neurofibrillary tangles and transitional forms were revealed in the treated cells by using antibodies either to tau (AT8) or TrpRS (FIG. 2a and FIG. 2b). (Mouse anti-human PHF-tau, clone AT8 against epitope containing phosphorylated Ser₂₀₂ was obtained from Innogenetics (Ghent, Belgium).)

A small number of such lesions were found in untreated or tryptamine-differentiated epithelial cells. This is consistent with a previous finding that neurofibrillary tangles occurred during healthy aging to a lesser extent than occurred in Alzheimer's disease brains. Price & Morris, Ann. Neurol. 45: 358-368 (1999). TrpRS-positive senile plaque-like manifestations were located also on the surface of the treated cells and in the extracellular space (FIG. 2a). FIG. 2c and FIG. 2d show such manifestations stained with antibodies to -amyloid (Frenkel et al., J. Neuroimmunol. 106: 23-31 (2000)) and Congo Red. Mouse anti- -amyloid precursor protein (APP) clone LN27 (recognizing an epitope within 1-200 amino acids) was obtained from Zymed (CA, USA).

To test whether TrpRS and tau co-localize in neurofibrillary tangles, fluorescent double labeling with AT8 and anti-TrpRS was performed. In acute-treated cells, the proteins were co-localized both in I-NFT-like encircled nuclei (FIG. 2e, f, i, and j) and E-NFT-like manifestations (FIG. 2g). In the perikarya of the control pretreated or untreated epithelial cells, few if any such formations were labeled (FIG. 2h and FIG. 2k). The staining is specific. The staining was absent in the control specimens probed with secondary antibodies (FIG. 2l). The TrpRS-depleted antibodies showed no reactivity. These results indicate that (a) tryptamine induces reversible neurodegeneration of Alzheimer's disease-type and (b) TrpRS associates with tryptamine-induced manifestations resembling neurofibrillary tangles and plaques.

To test whether tryptamine-induced manifestations are associated with neurofilaments, the cells were prepared either as a suspension (FIG. 2m) or as a monolayer and then analyzed by electron microscopy. Electron microscopy is performed as follows: Cells were washed and fixed as above and then collected with scraper (Greiner) or a rubber policeman. After centrifugation, the cells were first transferred to 0.1 M cacodylate buffer, pH 7.2, and then post-fixed in 1% OsO₄ in the same buffer for 1 hr. The cells were washed and then dehydrated, first in series of alcohols and then in propylenoxide. Specimens were polymerized in propilenoxide/Epon-812 or only Epon-812. Ultra-thin sections (70-90 nm) were placed on 300 mesh nickel (Ni) grids. For immunolabeling, some sections were washed with saturated methaperiodate. All the sections were blocked in 0.5% BSA, 1% gelatin, 1% Tween 20 and 1% glycine for 20 min. The sections were then incubated with antibodies overnight at 4° C. After washing, the sections were incubated with secondary antibodies conjugated with 10-nm gold (1:20) for 1 hr. Control specimens were incubated with secondary antibodies. The sections were dried, stained with 2% uranylacetate and lead citrate for 5 min and examined using electron microscopes Philips EM-410 and CM-12 at 100 kw.

In the treated cells, numerous neurofibrillary tangles were found that contain filaments of ˜10-15 nm in diameter. The tangles were composed either solely of straight filaments or a mixture of straight filaments and paired helical filaments and transitional forms, These results of this EXAMPLE were similar to previous findings in Alzheimer's disease brains. Papasozomenos, Lab. Invest. 60: 375-389 (1989).

The variety of neurofibrillary tangles forms might results tau isoforms assembling into either twisted paired helical filaments or straight filaments, when tau proteins contain either three or four repeats, respectively. Spilantini & Goedert, TINS 21: 429-433 (1998). In the tryptamine-treated cells, the bundles of filaments were gold labeled with antibodies to TrpRS and tau (FIG. 2m). However, staining with anti-TrpRS was more extensive than with AT8. Furthermore, in the treated cells, some neurofibrillary tangles failed to stain with phosphorylation-dependent AT8 antibody. These results indicate that neurofibrillary tangles formation does not result from the tau phosphorylation. The result of this EXAMPLE confirms previous in vitro findings that twisted filaments resembling Alzheimer's disease paired helical filaments can be assembled from both phosphorylated and non-phosphorylated tau. Spilantini & Goedert, TINS 21: 429-433 (1998).

No neurofibrillary tangles were revealed in untreated SH SY5Y cells. Other degenerative manifestations as autophagic vacuoles (AV) were also observed in tryptamine-treated cells (FIG. 2m). The autophagic vacuoles were composed of areas of the cytoplasm sequestered with single, double, or multiple membranes originating from the endoplasmic reticulum (ER). The membranes were seen in close association with paired helical filaments of about 20 nm in diameter and straight filaments surrounded by a fuzzy coat. Such autophagic vacuoles were observed in the experimental models of Creutzfeld-Jacob disease and scrapie. Liberski et al., Acta Neuropathol. 83: 134-139 (1992). It has been suggested that locally originated neurodegeneration, initially sequestered within the autophagic vacuoles, caused neuronal loss when diffuse. Accumulation of tau has been demonstrated in the autophagic vacuoles in chloroquine myopathy. Murakami et al., J. Neurophathol. Exp. Neurol. 57: 664-673 (1998). The membranes surrounding the autophagic vacuoles were intensely stained with antibody to TrpRS (inset, FIG. 2m). Numerous bodies showing ultrastructural variability were found in the extracellular space of the treated cells (FIG. 2m). The bodies showing ultrastructural variability appeared as packed bundles of parallel-oriented fibers; as areas of cytoplasm sequestered with membrane; or as bundles of amorphous material. The areas of cytoplasm included ribosomes and filaments decorated with anti-TrpRS.

The control specimens treated with gold-conjugated antibodies did not stain. These results indicate that tryptamine induces formation of (a) neurofibrillary tangles composed of paired helical filaments and straight filaments; (b) autophagic vacuoles associated with paired helical filaments; and (c) the presence of TrpRS in paired helical filaments of neurofibrillary tangles, membranes of autophagic vacuoles and in the extracellular space.

The involvement of tau and TrpRS in tryptamine-induced phenotypes was tested in extracts of control and treated cells. Triton extraction was performed as follows: Cells were lysed in 0.1 M Hepes buffer, pH 6.9, 0.5% Triton X100, 1 mM MgCl₂, 0.1 mM EDTA, 2 mM EGTA, 1 mM DTT, 1 mM PMSF, 0.4 mg/ml aprotinin, 0.1 mg/ml antipain, leupeptin, pepstatin, and hemastatin, 0.1 μM okadaic acid, 10 mM sodium ortovanadate, and 50 mM sodium fluoride for 20 min in situ at room temperature. The detergent-insoluble fraction was collected with scraper and washed in the same buffer excluding inhibitors of phosphatases. The detergent-insoluble fraction was then solubilized in SDS-sample buffer at 100° C. for 5 min, and spun.

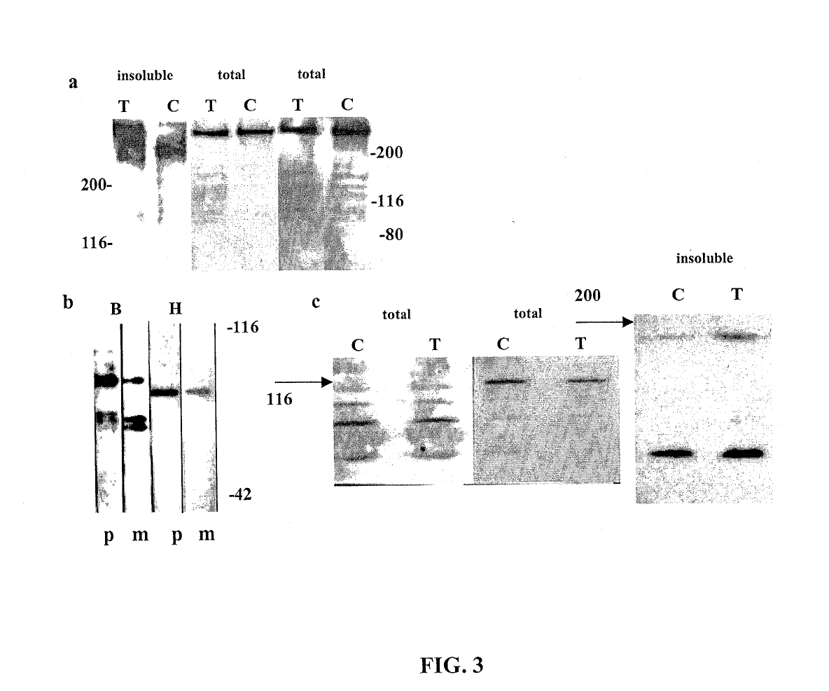

Phosphorylation of ˜210 kDa and 70 to 200 kDa tau forms was detected by Western blot with AT8 in the whole extracts of both control differentiated and acute-treated cells (right panels, FIG. 3a). In the detergent-insoluble fractions of the treated as well as control epithelial cells, this antibody reacts with two forms of ˜210 to 220 kDa (left lanes, FIG. 3a) and minor fragments of ˜70 and 35 kDa. The high molecular weight tau forms were detected earlier in neuronal cells as well as Alzheimer's disease brains. Gache et al., FEBS Lett. 272: 65-68 (1990). These data indicate that an abnormal phosphorylation of tau on Ser₂₀₂ that is shown in both control and treated cells is not sufficient for the neurofibrillary tangles formation in vivo.

To explore the cell state of TrpRS, the specificity of anti-TrpRS antibodies was examined. In non-denaturing ELISA and dot-blot, the anti-TrpRS monoclonal 6C10/C9, as well as 9D7 and polyclonal antibodies reacted with both bovine and human TrpRS. Bovine TrpRS was purified from bovine pancreas by the procedure of Paley, Eur. J. Biochem. 244: 780-788 (1997). Based on the human TrpRS gene sequence, the sense and anti-sense primers for RT-PCR were designed: 5′-ATGCCCAACAGTGAGCCCGCATCTCTG-3′ for the N-terminus and 5′-CTACTGAAAGTCGAAGGACAGCTTCCG-3′ for the C-terminus. PCR product was amplified with TaKaRa Ex Tag™ from a cDNA λgt10 phage library constructed from human fetal kidney (Clontech). The sequence and the size of the PCR product corresponded to human TrpRS gene. To introduce the Nde1 restriction sites on both termini and then to clone this gene into the overexpression vectors pET-11c and pET-14b (Novagen), reamplification of the PCR product was performed. The Nde1 restriction site-tagging primers were as follows: 5′-ACAGACCGACATATGCCCAACAGTGAGCCCGCA-3′ for the N-terminus and 5′-ACAACGTATCATATGCTACTDAAAGTCGAAGGACAG-3′ for the C-terminus. The 1420 base pair (bp) PCR product was purified, digested with vectors using Nde1 (NEB) and then ligated. E. coli XL-1 Blue cells were transformed with the plasmid DNA.

Plasmids from three clones, each containing the human TrpRS gene in the correct orientation as confirmed by BamH1 analysis, were chosen for subsequent transformation into BL-21 (DE-3, pLysS) and AD494 (DE-3) cells. Cell extracts were analyzed by SDS-PAGE and Coomassie blue staining before and after IPTG induction and by Western blot with antibodies to bovine TrpRS, using bovine TrpRS as a standard. The AD494 clone, which contains a pET11c plasmid highly expressing human TrpRS, was used in further experiments.

For Western blotting, dot-blotting and ELISA assays, the nitrocellulose or PVDF filters were blocked in 5% non-fat dry milk, incubated with AT8 or anti-TrpRS antibodies for 1 hr, washed with PBS, 0.1% Tween 20, treated with anti-mouse or anti-rabbit antibodies and then developed with ECL or TMB (KPL).

In the Western blot, both human recombinant ˜54 kDa and bovine ˜60 and ˜40-42 kDa (well-characterized proteolytic fragments) TrpRS reacted with 6C10/C9 and polyclonal antibodies (FIG. 3b), while poorly with 9D7. The main form and an additional high molecular weight TrpRS were induced in detergent-insoluble cytoskeleton by tryptamine treatment (right panel, FIG. 3c), whereas no difference was detected between acute-treated and pretreated cells (left and middle panels, FIG. 3c) when total extracts were analyzed. In the cytoskeleton of both treated and control cells, TrpRS was detected at a lower molecular weight than the cytoplasmic TrpRS (FIG. 3c). The 6C10/C9 antibody reacted more strongly with dimeric and trimeric TrpRS in re-probed blot (middle panel, FIG. 3c) in comparison with non-re-probed (left panel, FIG. 3c). The stripping and re-probing presumably provides conditions to renature TrpRS (see, Paley, Eur. J. Biochem. 244: 780-788 (1997)). These results indicate that (a) tryptamine induces high expression of cytoskeleton TrpRS forms and (b) both the 6C10/C9 and the 9D7 antibodies are generated against conformation-dependent epitopes on TrpRS.

To compare Alzheimer's disease-resembling manifestations induced by tryptamine in the cells with those occurred in diseased brain, a detailed immunohistological analysis of brain sections was conducted. Histochemistry was performed as follows: Autopsy specimens (4 μm sections of hippocampus, superior frontal gyrus and superior parietal lobule) from ten patients with neuropathologically confirmed diagnosis of Alzheimer's disease were obtained from the Mount Sinai Medical Center, NY. The tissues were fixed for two weeks in paraformaldehyde before paraffin processing. Non-demented brain sections from five patients were obtained from hospitals in Israel and Ukraine. Brains from 4 white Balb/c mice were fixed in 4% paraformaldehyde in PBS for 24 hr at 4° C. and in 10% formaldehyde in 0.9% NaCl for 48 hr at room temperature, washed and dehydrated. Paraffin-embedded 4 μm-sections were processed for immunostaining in parallel with patient's sections, deparaffinized in xylene, hydrated and incubated with 3% hydrogen peroxide in methanol for 30 min. at room temperature. After washing with PBS, sections were incubated with 0.1-0.3% Triton X-100 for 10-20 min., washed, blocked with 1% BSA for 30 min and then incubated with primary antibodies or supernatant of non-secretory hybridoma medium or control hybridoma medium containing 20% fetal calf serum overnight at 4° C. and then with peroxidase-conjugated antibodies for 1 hr at 37° C. and after with PAP in some labeling. Development was with DAB or 4-Cl-1-naphtol. The secondary antibodies overlie the control sections. Sections were counterstained with Highmann's Congo Red for 10 min. and Harris's hematoxylin for 2 min. For double labeling, sections were incubated with mixture of polyclonal anti-TrpRS and mAb 508 (1:50) or anti-tau overnight at 4° C. Mouse IgM mAb 508 was raised against residues 1-16 (Frenkel et al., J. Neuroimmunol. 106: 23-31 (2000)) and IgG monoclonal antibody 10D5 was raised to residues 1-28 of -amyloid (Athena Neuroscience CA, USA). Anti-tau mAb, clone T14, recognizing 83-120 of human tau, independent of phosphorylation state, was obtained from Zymed (CA, USA).

Successively, specimens were treated with anti-rabbit antibodies and developed with 4-cloro-1-naphtol (blue) and then with anti-mouse antibodies and developed with DAB (brown), mounted and examined under phase-contrast and polarized light.

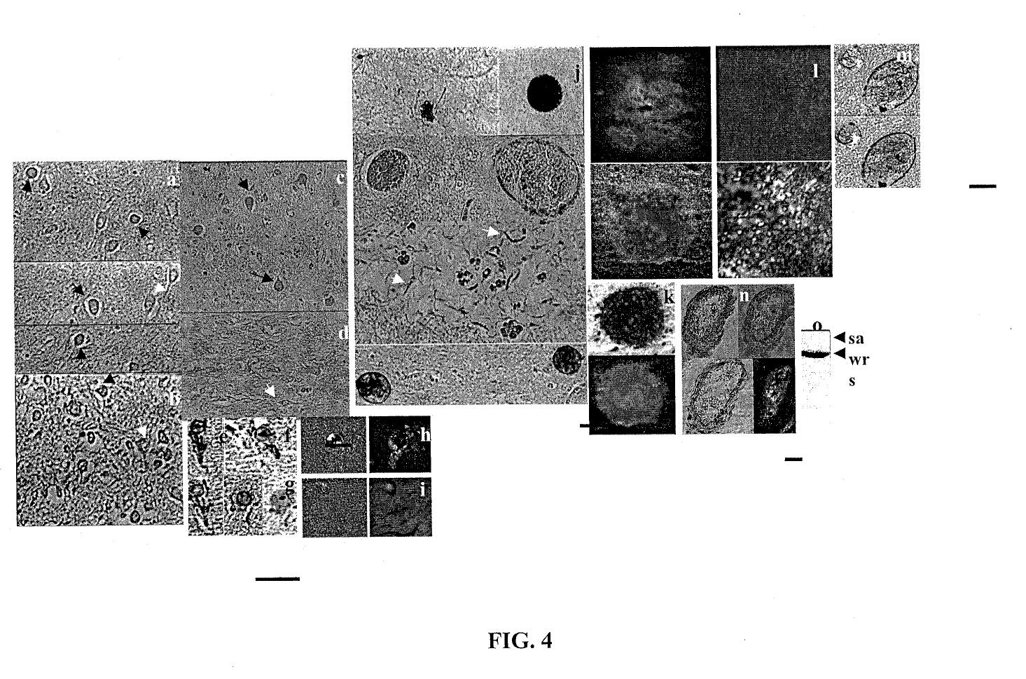

Numerous I-NFT (FIG. 4a-c) and E-NFT (FIG. 4e-h) were labeled with 6C10/C9 and 9D7 or polyclonal anti-TrpRS antibodies in the hippocampus, superior frontal gyrus and superior parietal lobule of Alzheimer's disease, but not in most neurons of non-demented patients (FIG. 4d). The intensive immunodecoration of TrpRS in contours of neurofibrillary tangles in Alzheimer's disease brain sections (FIG. 4a, c, and g) is similar to those in the tryptamine-treated cultured neurons (FIG. 2a). Confocal laser scanning microscopy of the optical sections confirmed an extracellular location of the E-NFT (FIG. 4h and FIG. 4i). Double labeling with antibodies to tau-protein and TrpRS showed intensely labeled I-NFT in Alzheimer's disease sections (FIG. 4b). TrpRS immunoreactivity has also been found in neuropil threads (FIG. 4j). Furthermore, numerous senile plaques with different morphologies were stained with 6C10/C9, 9D7 and polyclonal antiserum to TrpRS in the hippocampus, superior frontal gyrus and the superior parietal lobule of Alzheimer's disease brains (FIG. 4j). The TrpRS-positive senile plaques exhibited birefringence under polarized light following Congo Red staining (FIG. 4k). The structure of congophillic plaques was investigated using confocal laser scanning microscopy (FIG. 4l). In optical sections, TrpRS-type plaques showed a configuration consisting of irregularly arranged fibrous red fluorescent signals that seemed to correspond to many dystrophic neurites (left upper panel, FIG. 4l). The plaques observed under phase-contrast objective lenses (100×, oil immersion) have displayed structure consisting of compactly arranged “vesicles” which varied in size and shape with an average size of 0.6×0.9 μm. Many “vesicles” do not show red fluorescence (right panels, FIG. 4l) indicating that the plaques contain at least two different morphological compartments. In the senile plaques double-labeled with anti-TrpRS/ -amyloid, TrpRS is decorated predominantly in contours while -amyloid is diffusely distributed (FIG. 4m). The blood vessels were also double stained with antibodies to -amyloid and TrpRS (FIG. 4n). TrpRS was labeled in congophilic angiopathy (CA) of the vessels, preferably with polyclonal and less intensely with monoclonal antibodies, indicating the diversity of deposits in the senile plaques and vessels. This is consistent with a previous suggestion that a different pathogenesis exists in Alzheimer's disease than for either senile plaques or congophilic angiopathy. Verbeek et al., J. Neuropath. & Exp. Neurol. 56: 751-761 (1997). In non-demented brains no TrpRS-type plaques were stained with antibodies to TrpRS (FIG. 4d). Sections treated with set of control antibodies or hybridoma medium failed to show staining. The normal TrpRS distribution in the brain was followed in mice. Neither neurofibrillary tangles nor TrpRS-type senile plaques were labeled in the animal brain. The results indicate that TrpRS associates with the main Alzheimer's disease brain hallmarks and morphology of the lesions in Alzheimer's disease brains similar to those induced by tryptamine in neuronal cells.

To address whether TrpRS occurs in the extracellular space of tryptamine-treated cells and Alzheimer's disease brain due to secretion, Western blot of sera of Alzheimer's disease and non-demented patients was conducted. Human sera were prepared as follows: Sera were stored frozen at −20° C. Non-hemolyzed sera of patients (10 μl) were diluted 1:5 in 20 mM Tris-HCl, pH 6.8, incubated for 5 min. at 90° in SDS-sample buffer and subjected to 10-12% SDS-PAGE.

The ˜54 kDa protein was immunodetected in both Alzheimer's disease and non-demented patients sera using 6C10/C9 (FIG. 4o). The level of TrpRS detected with this antibody in sera was similar to that found in human total cell extracts, indicating that significant amount of TrpRS is secreted into sera.

Total cell extracts were performed as follows: Cells were washed with PBS and then either collected using cell scraper and then lysed or lysed in situ in SDS-sample buffer.

This EXAMPLE shows that a natural metabolite, minor biogenic amine tryptamine at the concentrations inhibiting the enzymatic activity of TrpRS (Paley et al., Exp. Cell Res. 195: 66-78 (1991)) led to neurodifferentiation of epithelial-like cells with transient neuron outgrowth and then to neurodegeneration with formation of intra- and extra-cellular manifestations similar to those found in the brains of Alzheimer's disease patients.

The biosynthesis of tryptamine occurs in mammalian cells by decarboxylation of tryptophan via the action of aromatic-L-amino acid decarboxylase (AAD). Monoamine oxidase (MAO) converts tryptamine to its metabolite indol-3-acetic acid. Tryptamine crosses the blood-brain barrier. The content of tryptamine in the brain is low, exhibiting the highest levels in the striatum and hippocampus, but it can be augmented by tryptophan administration or by inhibition of MAO. Mousseau et al., J. Neurochem. 62: 621-625 (1994); Mousseau & Butterworth, J. Neurochem. 63: 1052-1059 (1994). Peripheral tryptamine that originates from the kidney and liver where AAD activity is high, or from microbial metabolism in the intestine, might also increase its level in the brain. Significant amounts of tryptamine are contained in various foods and beverages. Tsuchiya et al., Biochem. Pharm. 50: 2109-2112 (1995). Tryptamine, a neuromodulator with behavioral effects, interacts with specific receptors in the human brain. Mousseau et al., J. Neurochem. 62: 621-625 (1994); Mousseau & Butterworth, J. Neurochem. 63: 1052-1059 (1994).

The association of TrpRS with Alzheimer's disease lesions can be explained by the canonical TrpRS role in protein biosynthesis and/or unusual properties. The machinery of eukaryotic protein synthesis including ARS was found associated with cytoskeleton. Condeelis, TIBS 20: 171-172 (1995); Melki et al., Biochemistry 30: 11536-11545 (1991); Mirande et al., Exp. Cell Res. 156: 91-102 (1985). Yeast lysyl-tRNA synthetases (KRS) and valyl-tRNA synthetases (VRS) bind to assembled tubulin of microtubules in vitro. Melki et al., Biochemistry 30: 11536-11545 (1991). KRS and VRS were displaced from microtubules by tau-peptide, which therefore binds to the same region on tubulin.

The findings of this EXAMPLE that TrpRS co-localizes with tau in neurofibrillary tangles and induces by tryptamine in cytoskeleton suggest that interaction between these proteins may lead to neurofibrillary tangles formation. Immunodecoration of TrpRS on the membranes of ER associated with paired helical filaments in autophagic vacuoles is consistent with the models for linking between intermediate filaments, microtubules, actin, myosin, and membrane-bound adhesion sites. Fuchs & Yang, Cell 98: 547-550 (1999). Studies of protein-synthesizing machinery in the axon compartment explain detection of TrpRS in axons. Koening & Giuditta, Neuroscience 89: 5115 (1999). In contrast to other housekeeping proteins, TrpRS was revealed in zymogenic secreted granules of bovine pancreas and as a secreted form in pancreatic juice (Favorova et al., Eur. J. Biochem. 184: 583-588 (1989)), as well as in the secretory salivary glands of Drosophila. Seshiah & Andrew, Mol. Biol. Cell 10: 1595-1608 (1999). Autoantibodies to TrpRS were found in human sera and the enzyme was immunoprecipitated from conditioned media of metabolically labeled HeLa cells. Paley et al., Immunol. Lett. 48, 201-207 (1995). TrpRS-associated kinase phosphorylates serum proteins (Paley, Eur. J. Biochem. 244: 780-788 (1997)), whereas serum factors stimulate phosphorylation of TrpRS. Recent data show that tyrosyl-tRNA synthetase is secreted under apoptotic conditions in human cells. Wakasugi & Schimmel, Science 284: 147-151 (1999). Thus, the results of this EXAMPLE indicate that TrpRS is a secreted protein and may contribute in senile plaque formation. The novel function of TrpRS and probably other ARS affirm these enzymes as pharmaceutical targets.

This EXAMPLE provides a physiologically relevant cellular model for the identification of compounds that interfere with neurofibrillary tangles and senile plaques formation. The conclusion supports the following simplified scheme: neuroactive biogenic amine→protein biosynthesis→neuronal differentiation→neurodegeneration.

### EXAMPLE II

Aszheimers's Disease Therapy Drug Platform—Blockage of Tryptamine Receptors for Alzeimer's Disease Treatment

The strategy of Alzheimer's disease drug therapy provided in this invention is based on using groups of effective and nontoxic antagonists and/or agonists for specific tryptamine receptors. The data that established the existence of specific tryptamine receptors on human brain cells was previously published. (Mousseau, D. D. & Butterworth, R. F., J. Neurochem. 63: 1052-1059 (1994)).

The tryptamine-reated human neuronal cells and animals bearing plaques and tangles serve as Alzheimer's disease models for screening for tryptamine receptor antagonists and/or agonists able for competing at the tryptamine binding sites. The affect of potential inhibitors of tryptamine binding will be examined using cytochemical, immunocytochemical, histochemical and immunohistochemical analysis of plaques and tangles in model cell and mice's/animals brain sections before and after treatment with potential drugs. The choice of appropriate compounds that are able to displace tryptamine is based on the published data of displacement of [³H]tryptamine specific binding to human frontal cortex by indolamines and other tryptophan metabolites, phenylethylamines, and miscellaneous drugs (Mousseau & Butterworth, J. Neurochemistry, 63, p. 1056, Table 3, (1994)). Based on the analysis of this data I concluded that methyl analogs of tryptamine, like (+)methyltryptamine, (+)dimethyltryptamine etc. as should be potent at displacing tryptamine from its binding site. The conclusion are based in data that (1) tryptamine was the most potent inhibitor of [³H]trypatmine binding to human cortical membrane preparation; (2) isomers of amphetamine and deprenyl exhibited higher affinity for the (+)-stereo isomer compared with (−)-isomer in binding to tryptamine site; (3) carboxylated tryptamine-tryptophan showed dramatically lower binding to trypatmine site than tryptamine; (4) 5-methoxytryptamine showed higher binding to tryptamine site compared with 5-hydroxytryptamine (serotonin, 5-HT); and (5) 5-hydroxytryptamine has >100 lower binding to tryptamine site than tryptamine. Thus, (+) isomer methyl- or dimethyltryptamine look promising for competing at tryptamine site. It has been published at <http://www.tryp.net> that dimethyltryptamine (DMT) is natural in human brain and a product of tryptamine metabolism. DMT is a psychedelic hallucinogen found in plants, animals, fungi, and frog. Tryptamine itself is not a hallucinogen. Note, the Alzheimer's disease symptoms are also hallucination or delusions.

Tryptamine-treated human neuronal cells are produced as described in EXAMPLE I or in published PCT international patent application WO 01/16286. Briefly, a human cultured neuroblast cell line is used. A particularly useful human neuroblast cell line is SH SY5Y (Biedler et al., Cancer Res. 33: 2643-2652 (1973)), but other human neuronal cells can be used as well may be used as well. The methods for creating the preferred cellular models of WO 01/16286 involve the selection of SH SY5Y epithelial cells, because such cells will, when differentiated, optimally exhibit the Alzheimer's characteristics for analysis. The SH SY5Y epithelial cells are either co-mingled with or isolated from the SH SY5Y neuroblast cells. SH SY5Y epithelial cells are treated with for a predetermined period of time and subsequently cultivated. In the case of the co-mingled epithelial cells, tryptamine exposure results in the preferential death of neuroblast cells and the treated epithelial cells expressing a differentiated neuron-axon phenotype. In the case of the isolated epithelial cells, tryptamine exposure tryptamine (50-100 micrograms/milliliter of tryptamine) results in the treated epithelial cells demonstrating neuronal characteristics with neurofibrillary tangles and changes of cytoskeleton-associated tryptophanyl-tRNA synthetase (TrpRS) protein expression.

The tryptamine treated animals are produced as described in EXAMPLE III.

### EXAMPLE III

Animal Model of Alzheimer's Disease

An Alzheimer's disease mouse model was obtained by using intravenous injections of male mice at 8 weeks or age- and sex-matched mice with 1-500 micrograms of tryptamine every second day for at least 2.5 weeks. In one embodiment, mice are injected with 50 micrograms of tryptamine for 4 months. However, the duration of treatment, intervals between injections and concentrations of tryptamine can be successfully varied. Control mice are injected with phosphate-buffered saline.

The tryptamine-treated mice appear to be healthy in comparison to the control mice. The treated mice are clean and fat. Indeed, the tryptamine-treated mice do not show any toxic effect during the four months of treatment. In contrast to the control mice, the treated mice do not show aggressive behavior.

Immunochemical assays with control and tryptamine-treated mice. Following tryptamine treatment, the brains of the treated model mice and the control untreated mice were routinely fixed; sectioned, stained and analyzed microscopically, using commonly used well-established methods. The mouse brains were examined for Alzheimer's-like manifestations.

Senile plaques, neurofibrillary tangles, and neuropil threads and fibrils were detected in different zones in the sections of brains of the treated mice following at least 2.5 weeks of treatment. These manifestations were not found in the control mice.

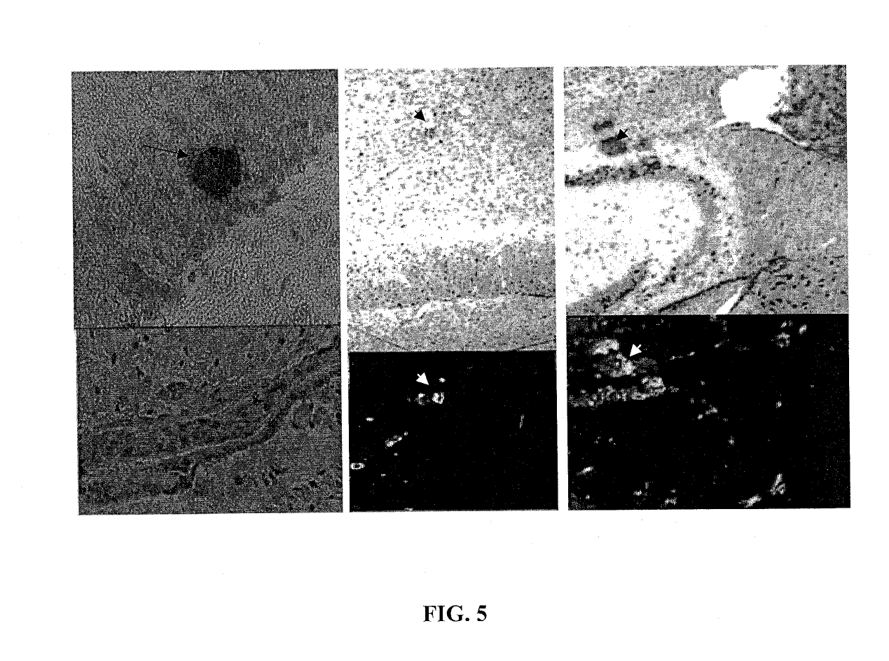

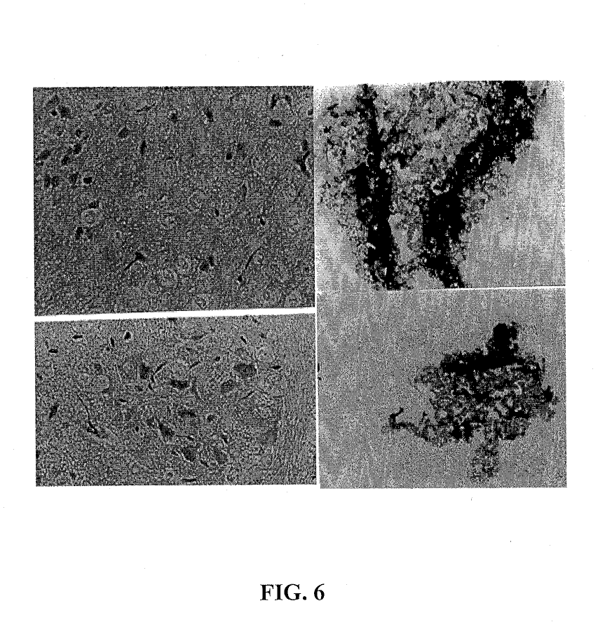

Microscope pictures were produced using an Olympus CK40) that demonstrate Congo Red staining and birefringence of senile plaques and specific Gallyas silver staining of neurofibrillary changes in the treated mouse model. FIG. 5 is a set of micrographs showing Congo Red staining and birefringence of sagittal sections from brains of tryptamine-treated mice. The arrow shows Congo Red-stained plaque in hippocampus, with the arrowhead pointing to identical deposits in Congo Red staining and birefringence. FIG. 6 is a set of micrographs showing silver staining of Alzheimer's neurofibrillary changes in the brain sections of tryptamine-treated mice.

Behavioral tests with control and tryptamine-treated mice. Behavioral assays with control and tryptamine-treated mice included tests for movement or coordination, sense of balance, orientation, and loss of interest. The behavioral tests in which the tryptamine-treated mice were tested included (a) a test of stable sitting (following 3 months of treatment), and (b) a test of running on a narrow stick (following 3, 4 and 6 months of the treatment). The results of both tests showed dramatic difference between control and treated mice. As shown in the testing this behavior of mice is age-dependent. Aged control mice showed slower running on the stick compare with younger mice. However in contrast to tryptamine-treated mine, control aged mice did not show halting. The data obtained regarding behavior of the control and tryptamine-treated mice are in good agreement with later signs of Alzheimer's disease compared with normal signs of aging (Source: Alzheimer's Disease: early warning signs and diagnostic resources. The Junior League of NYC, Inc, 1988). Normal aged people demonstrate increasing caution in movement and slower reaction times while Alzheimer'patients show visibly impaired movement or coordination, including slowing of movements, halting gait, reduced sense of balance, and frequent falls.

The model mice were made to run on a white metallic 80 cm stick having a diameter of 1 cm stick and placed 30 cm over the surface of a platform. Data in TABLE I are: (a) lag time (time that the mice were sitting at one end of the stick before beginning to run); (b) run time; and (c) the % of the stick that the mice successfully completed.

TABLE I

| | | | |
|---|---|---|---|
| | Lag time | Run time | |
| # | (seconds) | (seconds) | % of the way |

Control mice

| | | | |
|---|---|---|---|
| 1. | 0 | 33 | 100 |
| 2. | 0 | 22 | 100 |
| 3. | 0 | 53 | 50 |
| 4. | 0 | 20 | 10 (fell down) |
| 5. | 0 | 22 | 100 |
| 6. | 0 | 20 | 100 |
| 7. | 0 | 17 | 10 (fell down) |
| 8. | 0 | 15 | 100 |
| 9. | 0 | 6 | 100 |

Tryptamine-treated mice

| | | | |
|---|---|---|---|
| 1 | 50 | 2 | 40 |
| 2. | 47 | 0 | 0 |
| 3. | 11 | 0 | 0 |
| 4. | 17 | 0 | 0 |
| 5. | 13 | 0 | 0 |
| 6. | 40 | 10 | 20 |
| 7. | 3 | 17 | 50 |
| 8. | 9 | 0 | 0 |
| 9. | 3 | 0 | 0 |
| 10. | 15 | 0 | 0 |

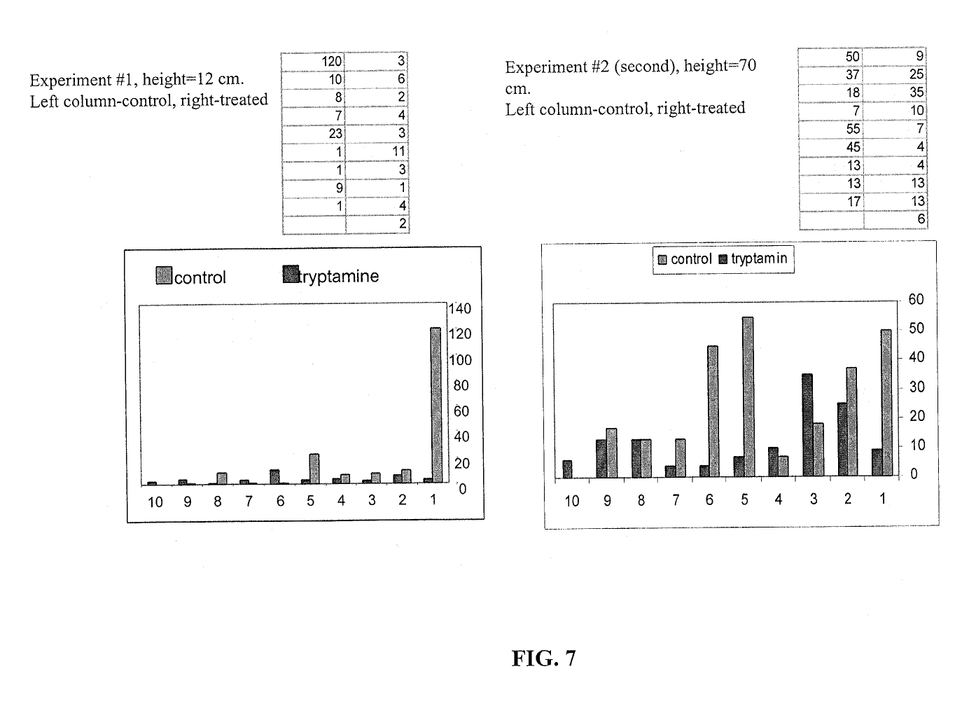

FIG. 7 is a set of tables and graphs showing measurements of the treated mice's stability The control and tryptamine-treated model mice were put on a narrow 1 cm diameter metallic stick. The time of their presence on the stick was measured in seconds. 66% of control versus 10% of treated mice showed stability for more than 6 seconds in the first experiment and 66% of control versus 20% of treated showed stability for more than 13 seconds in the second experiment. Thus, the model mice are 6.6 times less balanced than control.

The details of one or more embodiments of the invention are set forth in the accompanying description above. Although any methods and materials similar or equivalent to those described herein can be used in the practice or testing of the invention, the preferred methods and materials are now described. Other features, objects, and advantages of the invention will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All patents and publications cited in this specification are incorporated by reference.

The foregoing description has been presented only for the purposes of illustration and is not intended to limit the invention to the precise form disclosed, but by the claims appended hereto.

I claim:

1. An animal model of Alzheimer's disease, wherein:
(a) the animals have been treated with an amount of tryptamine sufficient to induce in the animals the morphological and behavioral characteristics of Alzheimer's disease; and
(b) the animals display the morphological and behavioral characteristics of Alzheimer's disease.

2. The animal model of claim 1, wherein the amount of tryptamine is 1-500 micrograms of tryptamine injected in a mouse every second day for at least 2.5 weeks.

3. The animal model of claim 1, wherein the amount of tryptamine is 50 micrograms of tryptamine injected in a mouse for 4 months.

4. The animal model of claim 1, wherein the morphological characteristics comprise the group consisting of neurofibrillary tangles and senile plaques.

5. The animal model of claim 1, wherein the behavioral characteristics comprise the group consisting of lack of stable sitting, and balance.

6. A method of generating an animal model of Alzheimer's disease, comprising the steps of;
administering to the animal an amount of tryptamine sufficient to induce in the animals the morphological and behavioral characteristics of Alzheimer's disease.

7. A method of identifying a substance that interacts with a neuron reducing Alzheimer's disease characteristics, comprising:
(a) identifying the morphological characteristics of Alzheimer's disease in a tryptamine-treated human differentiated neuronal cell;
(b) contacting the cell with a substance suspected of reducing Alzheimer's disease characteristics; and
(c) measuring the morphological characteristics of Alzheimer's disease in the contacted cell, wherein a reduced amount of morphological characteristics of Alzheimer's disease in the cell as compared with the un-contacted cell indicates that the substance is a substance that interacts with a neuron to reduce Alzheimer's disease characteristics.

8. A method of identifying a substance that prevents the accumulation of Alzheimer's disease characteristics, comprising:
(a) contacting a tryptamine-treated human differentiated neuronal cell with a substance suspected of preventing the accumulation of Alzheimer's disease characteristics;
(b) maintaining the contacted cell in culture; and
(c) measuring the morphological characteristics of Alzheimer's disease in the contacted cell, wherein a reduced amount of morphological characteristics of Alzheimer's disease in the contacted cell as compared with an un-contacted tryptamine-treated human differentiated neuronal cell that has been maintained in culture indicates that the substance is a substance that prevents the accumulation of Alzheimer's disease characteristics.

9. A kit for the detection of Alzheimer's disease, comprising:
(a) an anti-TrpRS antibody in a purified, lyophilized, ready-to-use form; and
(b) a positive control for detection by the anti-TrpRS antibody.

10. The kit of claim 9, wherein the anti-TrpRS antibody is a monoclonal antibody to TrpRS.

11. The kit of claim 9, wherein the anti-TrpRS antibody is a polyclonal antibody to TrpRS.

12. The kit of claim 9, wherein the positive control is human recombinant TrpRS protein.

13. The kit of claim 9, wherein the positive control is cell culture medium in which tryptamine-reated human neuronal differentiated cells had been cultured.
